SEQUENCE LISTING (12 sequences)

SEQ ID NO: 1 - DNA, 32 bp, artificial sequence
  primer
ggatccggtaccgagatggcgtttctccatgt

SEQ ID NO: 2 - DNA, 32 bp, artificial sequence
  primer
gagcttaagcttctgtctcaggtcagtatagg

SEQ ID NO: 3 - DNA, 21 bp, artificial sequence
  primer
agagcagactttctatttcac

SEQ ID NO: 4 - DNA, 19 bp, artificial sequence
  primer
ccatcttcacaggcatttc

SEQ ID NO: 5 - DNA, 19 bp, artificial sequence
  primer
tgacaaatacactgggaac

SEQ ID NO: 6 - DNA, 18 bp, artificial sequence
  primer
gccaatcttgtaggactg

SEQ ID NO: 7 - DNA, 21 bp, artificial sequence
  primer
aagacctctatgccaacacag

SEQ ID NO: 8 - DNA, 18 bp, artificial sequence
  primer
agccaccgatccacacag

SEQ ID NO: 9 - DNA, 24 bp, artificial sequence
  primer
agctactgtaatgatcagtcaacg

SEQ ID NO: 10 - DNA, 20 bp, artificial sequence
  primer
agaggtccttttcaccagca

SEQ ID NO: 11 - DNA, 19 bp, artificial sequence
  primer
gccttccgtgttcctaccc

SEQ ID NO: 12 - DNA, 18 bp, artificial sequence
  primer
tgcctgcttcaccacctt

(19) United States
(12) Patent Application Publication
(10) Pub. No.: US 2009/0075976 A1
(43) Pub. Date: Mar. 19, 2009

(54) Association between an anti-atherothrombotic and an angiotensin-converting enzyme inhibitor

(75) Inventors: Tony Verbeuren, Vernouillet (FR); Patricia Sansilvestri-Morel, Antony (FR); Alain Rupin, Savonnieres (FR); Marie-Odile Vallez, Montreuil (FR); Marie-Dominique Fratacci, Bois D'Arcy (FR); Laurence Lerond, Marly Le Roi (FR); Gilbert Lavielle, La Celle Saint Cloud (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, COURBEVOIE CEDEX (FR)

(21) Appl. No.: 12/231,981

(22) Filed: Sep. 8, 2008

(30) Foreign Application Priority Data
Sep. 11, 2007 (FR) 07.06345

(51) Int. Cl. A61K 31/55; A61K 31/18

(52) U.S. Cl. 514/212.07; 514/604

(57) ABSTRACT

The present invention relates to the association of an anti-atherothrombotic and an angiotensin-converting enzyme inhibitor (ACEI), and also to pharmaceutical compositions containing them, and to methods of treating vascular complications associated with diabetes, with atherothrombotic diseases, with hyperlipidaemia, with hypertension, with chronic venous diseases, with inflammation, with metabolic syndrome associated with obesity, or with cancer, with such association.

The present invention relates to the association of an anti-atherothrombotic and an angiotensin-converting enzyme inhibitor (ACEI), and also to pharmaceutical compositions containing them.

More specifically, the present invention relates to the association of a specific antagonist of TP receptors and an ACEI. Surprisingly, we have established that this association makes it possible to inhibit expression of the E-selectin gene, an adhesion molecule of 115 kDa involved in the mechanism of inflammation, E-selectin being in fact over-expressed in inflammatory tissues that are characteristic of various pathologies such as diabetes, atherothrombotic diseases, hypertension, obesity, Alzheimer's disease etc.

More precisely, E-selectin promotes the reversible adhesion between leukocytes and endothelial cells that constitutes an indispensable precondition for any inflammatory process (Frenette P. S. and Wagner D. D., Insights into selectin function from knockout mice, 1997, Thromb. and Haem., 78, 60-64). This step, referred to as “rolling”, necessitates the induction, at the surface of endothelial cells, of adhesion molecules from the selectin family (P-selectin (or CD62P) and E-selectin (or CD62E or ELAM, standing for “endothelial leukocyte adhesion molecule”)) which then interact with their leukocyte ligand (“sialylated carbohydrate ligand”, “P-selectin glycoprotein ligand 1” or PSGL1). Accordingly, the progression of leukocytes rolling on the endothelial wall of the vessels is slowed down. This is then followed by a step of firm adhesion, referred to as “sticking”, during which the leukocytes fix themselves to the surface of the vessel. Finally, the leukocytes migrate through the vessel wall towards the inflammatory tissues (diapedesis) via a gradient of chemotactic factors (TNF-α, IL-1, IL-8).

It is important to note that E-selectin expression is limited to the endothelium and responds to inflammatory stimuli such as IL-1, TNF-α or bacterial lipopolysaccharide (LPS). The level of E-selectin present at the surface of the cells is at a maximum 4 to 6 hours after stimulation. This period is long because it is synthesised de novo after stimulation of the cells. The level of E-selectin returns to its baseline level 24 hours after activation but, in vivo, in certain situations, E-selectin persists for longer at the surface of the cells.

Circulating forms of the various adhesion molecules, including E-selectin, exist. These soluble forms are probably generated by enzymatic cleavage at a site close to the insertion point at the membrane. The quantity of those soluble molecules correlates with the level of adhesion molecules present at the surface of the endothelial cells (Leeuwenberg J F M, Smeets E F, Neefjes J J et al, E-selectin and intercellular adhesion molecule-1 are released by human endothelial cells in vitro, 1992, Immunology, 77, 543-549). Accordingly, an increase in the circulating levels of soluble adhesion molecules, and more particularly E-selectin, indicates activation of the endothelial cells (Smith C W, Potential significance of circulating E-selectin, 1997, Circulation, 95, 1986-1988).

An increase in the level of plasma E-selectin has been established in obesity and a positive correlation with body mass index has been demonstrated (Ferri C, Desideri G, Valenti, et al, Early upregulation of endothelial adhesion molecules in obese hypertensive men, 1999, Hypertension, 34, 568-573). Increased oxidative stress in the cardiovascular system of those patients and/or metabolic stimuli such as, for example, insulin in contact with the endothelium might explain those observations. A correlation between the level of E-selectin and vascular risk is, in fact, also present in diabetic patients suffering from type I or II diabetes (Bannan S, Mansfield M W, Grant P J, Soluble vascular cell adhesion molecule-1 and E-selectin levels in relation to vascular risk factor and to E-selectin genotype in the first degree relatives of NIDDM patients and in NIDDM patients, 1998, Diabetologia, 41, 460-466). The level of E-selectin is moreover considered to be a marker for the vascular complications associated with diabetes. Insulin resistance, hyperglycaemia and hyperinsulinaemia increase E-selectin expression, thereby explaining the predisposition to atherosclerosis observed in those patients.

An increase in the levels of circulating E-selectin has been widely found in hyperlipidaemic patients, with a reduction in those levels after hypolipidaemic treatment (Hackman A, Abe Y, Insull W et al, Levels of soluble cell adhesion molecules in patients with dyslipemia, 1996, Circulation, 93, 1334-1338). This suggests that cholesterol levels influence the level of soluble E-selectin. Severe dyslipidaemia does in fact bring about endothelial dysfunction, with increased E-selectin expression in the endothelial cells.

Numerous studies have shown correlations between the level and expression of E-selectin and hypercholesterolaemia and atherosclerosis. In view of the fact that E-selectin synthesis is induced by cytokines, an increase in the level of E-selectin could be a marker of vascular inflammation.

Numerous studies have also demonstrated, in patients suffering from chronic venous diseases, activation of the endothelial cells due to venous hypertension and therefore an increase in the circulating levels of adhesion molecules (Saharay M, Shields D A, Georgiannos S N et al, Endothelial activation in patients with chronic venous disease, 1998, Eur J Vasc Surg, 15, 342-349; Verbeuren T J, Bouskela E, Cohen R A et al, Regulation of adhesion molecules: a new target for the treatment of chronic venous insufficiency, 2000, Microcirculation, 7, S41-S48).

Furthermore, increased E-selectin levels have also been described in hypertensive patients (Ferri C, Bellini C, Desideri G et al, Clustering of endothelial markers of vascular damage in human salt-sensitive hypertension. Influence of dietary sodium load and depletion, 1998, Hypertension, 32, 862-868).

Similarly, numerous references in the literature describe the role of E-selectin in complications of the renal system (Singbartl K, Ley K, Protection from ischemia-reperfusion induced severe acute renal failure by blocking E-selectin, 2000, Crit. Care. Med., 28(7), 2507-2514, Nakatani K, Fujii H, Hasegawa H et al, Endothelial adhesion molecules in glomerular lesions: association with their severity and diversity in lupus models, 2004, Kidney Int., 65(4), 1290-1300).

The involvement of E-selectin in the case of patients suffering from Alzheimer's disease has also been demonstrated, raised E-selectin levels having in fact been observed in those patients (Borroni B, Volpi R, Martini G, Del Bono R, Archetti S, Colciaghi F, Akkawi N M, Di Luca M, Romanelli G, Caimi L, and Padovani A, Peripheral blood abnormalities in Alzheimer Disease: Evidence for early endothelial dysfunction, 2002, Alzheimer's disease and Associated disorders, 16 (3), 150-155).

Finally, numerous publications demonstrate the involvement of E-selectin levels in metastatic processes and therefore in cancer (Kobayashi H, Boelte K C, Lin P C, Endothelial Cell Adhesion Molecules and Cancer Progression, 2007, Current Medical Chemistry, 14, 377-386; Kneuer C, Ehrhardt C, Radomski M W, Bakowsky U, Selectins—potential pharmacological targets?, 2006, Drug Discovery Today, Vol. 11, N^{o} 21/22, 1034-1040).

The present invention relates to the association of an anti-atherothrombotic and an angiotensin-converting enzyme inhibitor (ACEI) wherein:
the anti-atherothrombotic compound is a compound (A) of formula (I):

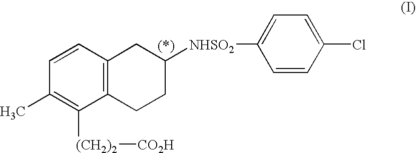

in racemic form or in the form of an optically pure isomer, or a pharmaceutically acceptable addition salt thereof. Described in the patent specification EP 648741, this compound is a potent antagonist of TP receptors, more particularly a specific antagonist of thromboxane A₂ and prostaglandin-endoperoxide receptors (PGG₂-PGH₂). It has moreover been shown that this compound significantly reduced the endothelial expression of the adhesion molecule VCAM-1 in diabetic atheromatous Apo E^{-/-} mice (Zuccollo A, Shi C, Mastroianni R et al, The thromboxane A2 receptor antagonist S 18886 prevents enhanced atherogenesis caused by diabetes mellitus, 2005, Circulation, 112, 3001-3008).
the angiotensin-converting enzyme inhibitor (ACEI) is selected, without implying any limitation, from the following compounds: perindopril, optionally in the form of its active metabolite perindoprilate, ramipril, optionally in the form of its active metabolite ramiprilate, enalapril, optionally in the form of its active metabolite enalaprilate, captopril, lisinopril, delapril, fosinopril, quinapril, spirapril, imidapril, trandolapril, optionally in the form of its active metabolite trandolaprilate, benazepril, cilazapril, temocapril, alacepril, ceronapril, moveltipril and moexipril, and also addition salts thereof with a pharmaceutically acceptable acid or base. It may be noted that certain ACEIs inhibit induction of the expression of adhesion molecules in the endothelial cells of Apo E^{-/-} mice infused with angiotensin II (da Cunha V, Tham D M, Martin-McNulty B et al, Enalapril attenuates angiotensin II-induced atherosclerosis and vascular inflammation, 2005, Atherosclerosis, 178, 9-17).

By studying the interaction between the thromboxane A₂-TP receptor and renin-angiotensin systems, we have demonstrated a substantial synergy of those pathways with regard to the expression of adhesion molecules.

For that purpose, an antagonist of TP receptors was used at a concentration having no effect on the expression of E-selectin induced by TNF-α in human endothelial cells. That same concentration was then tested in the presence of various similarly inactive concentrations of several ACEIs. A significant reduction in the E-selectin expression induced by TNF-α in human endothelial cells was then observed with the association of the two compounds, demonstrating a synergy between the two compounds which could not have been foreseen.

This synergistic effect has also been demonstrated in a thrombosis and arterial pressure test in the rat. In the course of that test it was shown that the antithrombotic activity of the compound (A) of formula (I) is potentiated in the presence of the compound (B) and increases in extremely substantial and entirely unforeseeable manner. This test also demonstrates that the presence of the compound (A) of formula (I) potentiates the anti-hypertensive effect of the compound (B) in substantial and unforeseeable manner.

The association of the compound (A) of formula (I) and the compound (B) has also made it possible to clearly and substantially reduce the expression of vascular cell adhesion molecule 1 (VCAM-1) in the aorta and of fibronectin in the kidney in a model of diabetic atheromatous mice.

These results make it possible to envisage the use of an association [TP receptor antagonist/ACEI] in the manufacture of a medicament for use in the treatment of vascular, and more particularly cardiovascular and cerebrovascular, complications associated with diabetes, with atherothrombotic diseases, with hyperlipidaemia, with hypertension, with chronic venous diseases, with inflammation, with metabolic syndrome associated with obesity, or with cancer. Among the atherothrombotic diseases, the compositions according to the invention are especially useful in the treatment of myocardial infarction, angina pectoris, cerebral vascular accidents, aortic aneurysms or arteritis of the lower limbs. Furthermore, the nephropathy associated with diabetes, with hypertension or with inflammatory diseases is also an indication in which the association [TP receptor antagonist/ACEI] is especially useful. Finally, diabetic retinopathy also belongs among the preferred therapeutic indications of this invention.

Vascular risk factors and vascular diseases such as hypertension, obesity, diabetes, cardiac diseases, cerebrovascular diseases and hyperlipidaemia and therefore atherosclerosis are involved in the genesis of dementias such as Alzheimer's disease and vascular dementia (Qiu C., De Ronchi D. and Fratiglioni L., The epidemiology of the dementias: an update, 2007, Current Opinion in Psychiatry, 20, 380-385,). Moreover, in neurodegenerative diseases such as Alzheimer's disease, an increase in isoprostane levels has been observed. These isoprostanes are markers, but also mediators, of the oxidative stress which could lie at the origin of the disease (Montuschi P., Barnes P J. and Jackson Roberts II L., Isoprostanes: markers and mediators of oxidative stress, 2004, The FASEB Journal, 18, 1791-1800). The isoprostanes exert at least part of their activity by stimulating the TP receptors (Montuschi P., Barnes P J. and Jackson Roberts II L., Isoprostanes: markers and mediators of oxidative stress, 2004, The FASEB Journal, 18, 1791-1800) and their activity would therefore be blocked by the present association.

As demonstrated by the studies described in the present patent application, our association acts on the vascular diseases which are risk factors for dementias.

Preferred ACEIs are perindopril of formula (B) and ramipril of formula (C), and also salts thereof, more especially perindopril of formula (B) and salts thereof:

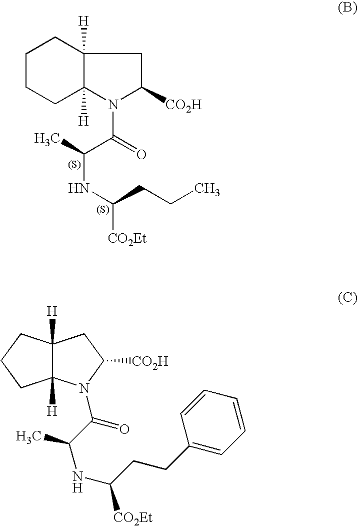

Among the addition salts of perindopril, there may be mentioned, without implying any limitation, addition salts with a pharmaceutically acceptable base such as the salts of tert-butylamine, arginine, sodium, potassium etc.

Perindopril will preferably in the form of a tert-butylamine salt or an arginine salt.

In the associations according to the invention, the compound (A) is preferably 3-[(6R)-6-[[(4-chlorophenyl)sulphonyl]amino]-2-methyl-5,6,7,8-tetrahydronaphth-1-yl]propanoic acid, also known as terutroban. Analogous associations involving other TP receptor antagonists such as ifetroban or ramatroban can also be envisaged.

Among the addition salts of the compound (A), there may be mentioned, without implying any limitation, addition salts with a pharmaceutically acceptable base such as the salts of sodium, potassium, tert-butylamine, diethylamine etc. The sodium salt of terutroban will be more especially preferred.

In the pharmaceutical compositions according to the invention, the amounts of ACEI and of TP receptor antagonist are matched to the nature of these active ingredients, and their relative proportions are accordingly variable as a function of the active ingredients.

When the compound (A) is terutroban in the form of the sodium salt and the ACEI is perindopril in the form of the tert-butylamine or arginine salt, those proportions are from 10 to 40% of the total weight of the active ingredients for perindopril and from 60 to 90% of the total weight of the active ingredients for terutroban.

The preferred percentages for that association are from 15 to 25% perindopril in the form of the tert-butylamine salt as against from 75 to 85% terutroban in the form of the sodium salt, and from 20 to 30% perindopril in the form of the arginine salt as against from 70 to 80% terutroban in the form of the sodium salt.

The present invention relates also to pharmaceutical compositions comprising an association of the compound (A) and an ACEI, one or both optionally in the form of pharmaceutically acceptable salts, with one or more appropriate, inert, non-toxic carriers or excipients.

In the pharmaceutical compositions according to the invention, the weight proportion of active ingredients (weight of active ingredients over the total weight of the composition) is from 5 to 50%.

As regards the pharmaceutically acceptable excipients, there may be mentioned, without implying any limitation, binders, diluents, disintegrating agents, stabilisers, preservatives, lubricants, fragrances, aromas or sweeteners.

Among the pharmaceutical compositions according to the invention, there will be more especially selected those that are suitable for administration by the oral, parenteral and especially intravenous, per- or trans-cutaneous, nasal, rectal, perlingual, ocular or respiratory routes, more specifically tablets or dragées, sublingual tablets, hard gelatin capsules, glossettes, capsules, lozenges, injectable preparations, aerosols, eye drops, nose drops, suppositories, creams, ointments, dermal gels etc.

The preferred route of administration is the oral route and the corresponding pharmaceutical compositions may allow instantaneous or deferred release of the active ingredients.

Preferred pharmaceutical compositions are tablets.

The unit dose can be varied according to the nature and severity of the disorder, the administration route and also the age and weight of the patient. In the compositions according to the invention it ranges from 1 to 100 mg for the compound (A) and from 0.5 to 100 mg according to the nature of the ACEI per 24 hours in one or more administrations. When the ACEI is perindopril, the daily dose administered is from 0.5 to 20 mg in one or more administrations.

The Examples of compositions hereinbelow are given without implying any limitation.

### Terutroban/Perindopril Tablets:

### EXAMPLE 1

| Constituents | Amount (mg) |
|---|---|
| terutroban, sodium salt | 30 |
| perindopril, tert-butylamine salt | 8 |
| hydrophobic colloidal silica | 0.4 |
| starch | 6 |
| magnesium stearate | 2 |
| microcrystalline cellulose | 50 |
| lactose | 103.6 |
| For a tablet totalling | 200 |

### EXAMPLE 2

| Constituents | Amount (mg) |
|---|---|
| terutroban, sodium salt | 30 |
| perindopril, arginine salt | 10 |
| hydrophobic colloidal silica | 0.4 |
| starch | 6 |
| magnesium stearate | 2 |
| microcrystalline cellulose | 50 |
| lactose | 101.6 |
| For a tablet totalling | 200 |

### Pharmacological Results

### In Vitro Inhibition of E-Selectin Expression

1) Cell Culture

The study is carried out on human endothelial cells HUVEC (Human Umbilical Vein Endothelial Cells, Clonetics Co). The cells are cultured in an EBM2 medium (Endothelial Basal Medium, Clonetics Co) supplemented with 2% FCS (Fœtal Calf Serum) and EGM2 (Endothelial Growth Medium, Clonetics Co).

2) E-Selectin Promoter Cloning

### a) Amplification of the Promoter by PCR

An 850 bp fragment, corresponding to the human E-selectin promoter, extending from the nucleotides at positions −800 to +50 (accession no. M64485; Tamaru et al., E-selectin gene expression is induced synergistically with the coexistence of activated classic protein kinase C and signals elicited by interleukin-1β but not tumor necrosis factor-α; 1999, J. Biol. Chem, 274, 3753-3763), was amplified by PCR and sub-cloned. In a final reaction volume of 100 μl, 5 units of native Pyrococcus furiosus (Pfu) DNA polymerase (Stratagene) were placed in the presence of 1 μg of human genomic DNA (Clontech), 200 μM of dNTP (deoxynucleotide triphosphate) (Clontech) and 200 ng of primers in a medium containing the buffer specific to the enzyme. The set of primers used is as follows:

(SEQ ID NO.1)

5′-GGATCCGGTACCGAGATGGCGTTTCTCCATGT
and

(SEQ ID NO.2)

5′-GAGCTTAAGCTTCTGTCTCAGGTCAGTATAGG

The PCR program, on a Gene Amp PCR system 9700 apparatus, comprises initiation at 94° C. for 1 min and then an amplification over 35 cycles (94° C. for 1 min, 55° C. for 1 min, 72° C. for 3 min). The PCR product is then precipitated overnight at −20° C. in the presence of 0.3M sodium acetate pH 5.2 and ethanol. After centrifuging at 14000 rpm at 4° C., the sediment is re-suspended in ethanol 70% and again centrifuged at 14000 rpm at 4° C. The sediment obtained is then dried and subsequently taken up in water.

### b) Digestion of the Promoter Sequence

The amplified sequence is then digested in two steps by the restriction enzymes Kpn I and Hind III. The amplified sequence is digested for 1 hour 30 minutes at 37° C. in the presence of 30 units of enzyme and 100 μg/ml of BSA (Bovine Serum Albumin). After each digestion, the product obtained is systematically purified on Micro Bio-Spin® Chromatography columns (Bio-Rad) in order to remove the buffer salts.

### c) Construction of the PGL3/E-Selectin Plasmid

The pGL3 Basic plasmid (Promega), containing the luciferase gene of the firefly, is digested by the restriction enzymes Kpn I and Hind III in accordance with the same protocol as for the insert and is then purified on Low Melting 1% agarose gel.

Ligation of the pGL3-Basic plasmid vector and the insert corresponding to the E-selectin promoter was carried out using T4 DNA Ligase (LigaFast™ Rapid DNA Ligation System from Promega). Conventionally, during ligation, an excess of insert that is equivalent to three times the amount of vector is used. Moreover, because this insert is a sixth of the length of the vector (0.85 kb as opposed to 4.8 kb), maintaining stoichiometric equilibrium requires six times the mass of vector. 10.6 ng of insert and 25 ng of pGL3 Basic vector were therefore reacted in the presence of 3 units of T4 ligase, at ambient temperature.

3) Transfection of HUVEC Cells

The PGL3/E-selectin plasmid is transfected into HUVEC cells before the fourth passage. Transfection is carried out in plates having cells at 50% confluence, depositing Lipofectin® (Invitrogen) and 3 μg of PGL3/E-selectin plasmid in each of the wells. The Lipofectin® (6 μg/ml) is previously activated for 30 minutes in an OPTI-MEM medium (GIBCO™) and then brought into contact for 15 minutes with the plasmids previously diluted with the medium. The cells are incubated for 4 hours at 37° C. in an atmosphere containing 5% CO₂ and 95% O₂. The transfection medium is withdrawn and replaced overnight by an enriched culture medium in order to stabilise the cells.

Expression of the reporter genes is induced over 4 hours in an M199 medium without serum (GIBCO™). The cells are scratched and lysed in a lysis buffer (Dual-Luciferase® kit, Promega) and then held at −20° C.

The induction phase is 4 hours for the HUVEC cells in the presence of 100 U/ml of Tumour Necrosis Factor-α (TNF-α). During that induction phase, there are added different concentrations of, on the one hand:
perindoprilate (0 to 100 M), terutroban sodium salt (0 to 100 μM), or terutroban sodium salt (30 μM)+different concentrations of perindoprilate (10, 30 and 100 μM) (Table 1) and, on the other hand:
ramiprilate (0 to 100 μM), terutroban sodium salt (0 to 100 μM), or terutroban sodium salt (10 μM)+different concentrations of ramiprilate (30 and 100 μM) (Table 2).

4) Determination of the Promoter Activity

The E-selectin promoter activity is determined by quantification of the luciferase activity produced (Dual-Luciferase® kit, Promega). A solution of luciferin, the substrate of the firefly luciferase, is added to each well. This results in an emission of light. The plate is incubated for 10 minutes in the dark because the luciferase activity is light-sensitive, and then a luminometer reading is started in order to quantify the photons emitted (Wallac, Perkin Elmer), the result obtained being the average cpm (counts per minute) over a period of 5 seconds.

5) Results for Terutroban-Perindopril

Terutroban (compound A) in the form of the sodium salt and perindopril (compound B) in the form of its active metabolite perindoprilate were tested separately at different concentrations (0, 10, 30, 100 μM) on the HUVEC cells after induction with TNF-α. In analogous manner, compound A in the form of the sodium salt (30 μM)+different concentrations of compound B in the form its active metabolite (0, 10, 30, 100 μM) were studied. The activity of the E-selectin promoter is measured under control conditions and in the presence of products in the induced state. The activities, expressed in cpm and as a percentage of the control observations, are illustrated in Table 1.

TABLE 1
Measurement of the activity of the E-selectin promoter in the
presence of the sodium salt of compound A, the active metabolite
of compound B, or the sodium salt of compound A (30 μM) + the
active metabolite of compound B under TNF-α-induced
conditions (100 U/ml).

| | | Activity in cpm | |
|---|---|---|---|
| | | %/Control | % |
| | | MEAN ± SDM | inhibition/control |
| Compound A, | Control | 100% | |
| sodium salt | 10 μM | 105.8 ± 7.9 | |
| | 30 μM | 99.5 ± 9.1 | |
| | 100 μM | 88.8 ± 8.9 | |
| Compound B, active | Control | 100% | |
| metabolite | 10 μM | 99.5 ± 7.8 | |
| | 30 μM | 100.3 ± 9.2 | |
| | 100 μM | 82.8 ± 11.5 | |
| Compound A, | Control | 100% | |
| sodium salt (30 μM) + | 10 μM | 60.8 ± 8.1 (**) | 41.7 ± 10.1 |
| Compound B, active | 30 μM | 67.1 ± 6.0 (*) | 35.4 ± 5.0 |
| metabolite | 100 μM | 47.6 ± 8.7 (**) | 52.5 ± 8.5 |

(*) p < 0.05;
(**) p < 0.01 relative to the control single-factor ANOVA, with post-test Dunnett (n = 4-5).

Terutroban sodium salt and perindopril in the form its active metabolite perindoprilate have no effect on expression of the E-selectin gene at the concentrations tested. When perindoprilate is co-incubated with terutroban sodium salt (30 μM), inhibition of expression of the E-selectin gene is then observed from 10 μM perindoprilate (60.8% activity of expression of the E-selectin gene versus 100% without product; p<0.01, single-factor ANOVA, with post-test Dunnett).

The results show very clearly that administration of these two compounds in association makes it possible to obtain a substantial synergistic effect which was entirely unexpected.

6) Results for Terutroban-Ramipril

Terutroban (compound A) in the form of the sodium salt and ramipril (compound C) in the form of its active metabolite ramiprilate were tested separately at different concentrations (0, 30, 100 μM) on the HUVEC cells after induction with TNF-α. In analogous manner, compound A in the form of the sodium salt (10 μM)+different concentrations of compound C in the form its active metabolite (0, 30, 100 μM) were studied. The activity of the E-selectin promoter is measured under control conditions and in the presence of products in the induced state. The activities, expressed in cpm and as a percentage of the control observations, are illustrated in Table 2.

TABLE 2
Measurement of the activity of the E-selectin promoter in the
presence of the sodium salt of compound A, the active metabolite
of compound C, or the sodium salt of compound A (10 μM) + the
active metabolite of compound C under TNF-α-induced
conditions (100 U/ml).

| | | Activity in cpm | |
|---|---|---|---|
| | | %/Control | |
| | | MEAN ± SDM | % inhibition/control |
| Compound A, | Control | 100% | |
| sodium salt | 30 μM | 99.5 ± 9.1 | |
| | 100 μM | 88.8 ± 8.9 | |
| Compound C, | Control | 100% | |
| active metabolite | 30 μM | 111.1 ± 17.2 | |
| | 100 μM | 114.6 ± 38.9 | |
| Compound A, | Control | 100% | |
| sodium salt (10 μM) + | 30 μM | 55.2 ± 4.8 (**) | 44.7 ± 4.8 |
| Compound C, | 100 μM | 44.2 ± 9.1 (**) | 55.8 ± 9.1 |
| active metabolite | | | |

(**) p < 0.01 relative to the control single-factor ANOVA, with post-test Dunnett (n = 3).

Terutroban in the form of the sodium salt and ramipril in the form its active metabolite ramiprilate have no effect on expression of the E-selectin gene at the concentrations tested. When ramiprilate is co-incubated with terutroban sodium salt (10 μM), inhibition of expression of the E-selectin gene is then observed from 30 μM ramiprilate (55.2% activity of expression of the E-selectin gene versus 100% without product; p<0.01, single-factor ANOVA, with post-test Dunnett).

The results show very clearly that administration of these two compounds in association makes it possible to obtain a substantial synergistic effect which was entirely unexpected.

The results for the association of, on the one hand, terutroban (compound A) in the form of the sodium salt and perindopril (compound B) in the form of its active metabolite and, on the other hand, terutroban (compound A) in the form of the sodium salt and ramipril (compound C) in the form of its active metabolite show that the association between compound (A) of formula (I) and an angiotensin-converting enzyme inhibitor has a substantial synergistic effect which was entirely unexpected.

### Inhibition of Thrombosis and Arterial Pressure in the Rat

1) Equipment and Methods

The thrombosis technique used is that of Tanaka and co-workers (Eur. J. Pharmacol., 2008; 401, 413-18).

a) Arterial Thrombosis

CD rats (350-375 g) are anaesthetised using pentobarbital 50 mg/kg IP and are placed on a thermostatically controlled blanket. After laparotomy, the aorta is exposed. Thrombosis is induced by setting in place a pellet of filter paper (8 mm) saturated with FeCl₃ 50% for 10 minutes. 20 minutes after removal of the pellet, the artery is ligated and incised; the clot formed is weighed. Some animals are treated with terutroban (compound A) in the form of the sodium salt at a dose of 0.1 mg/kg, others with perindopril (compound B) in the form of the tert-butylamine salt at a dose of 1 mg/kg, and others with terutroban in the form of the sodium salt (compound A) at a dose of 0.1 mg/kg and perindopril (compound B) in the form of the tert-butylamine salt at 1 mg/kg.

b) Arterial Pressure

After anaesthesia using pentobarbital (50 mg/kg IP), the animal is placed on a thermostatically controlled blanket. The carotid artery is exposed and a catheter is introduced in order to monitor the arterial pressure with the aid of a Gould sensor connected to AcqKnowledge software. The arterial pressure is recorded 1 hour after treatment, for a period of 30 minutes. Some animals are treated with terutroban (compound A) in the form of the sodium salt at a dose of 0.1 mg/kg, others with perindopril (compound B) in the form of the tert-butylamine salt at a dose of 0.3 mg/kg, and others with terutroban (compound A) in the form of the sodium salt at 0.1 mg/kg and perindopril (compound B) in the form of the tert-butylamine salt at 0.3 mg/kg.

2) Results

a) Arterial Thrombosis

The weights of clots in the control rats are 17.1±1.3 mg; treatment with terutroban (compound A) sodium salt at 0.1 mg/kg did not alter that weight: 16.8±1.3 mg. The weights of clots in the control rats are 15.6±0.7 mg; treatment with perindopril (compound B) tert-butylamine salt at 1 mg/kg did not alter that weight: 15.2±1.1 mg. The weights of clots in the control rats are 16.3±0.8 mg; treatment with the association of terutroban (compound A) sodium salt at 0.1 mg/kg with perindopril (compound B) tert-butylamine salt at 1 mg/kg significantly reduced the weight of the clot to 10.1±0.6 mg. These results demonstrate the unexpected synergy of action of the two substances in respect of arterial thrombosis.

b) Arterial Pressure

The arterial pressure of the control rats is 131±7 mmHg. Neither terutroban (compound A) sodium salt at 0.1 mg/kg nor perindopril (compound B) tert-butylamine at 0.3 mg/kg (which is an inactive dose in the rat) altered that pressure: 126±7 mmHg and 120±9 mmHg, respectively. The association of terutroban (compound A) sodium salt and perindopril (compound B) tert-butylamine salt markedly and significantly reduced arterial pressure to 95±7 mmHg.

These results demonstrate the unexpected synergy of action of the two substances in respect of regulation of arterial pressure.

This test demonstrates the inhibitory activities in respect of thrombosis and arterial pressure of the association of terutroban (compound A) and perindopril (compound B) and accordingly illustrates the potential for the treatment, using this association, of arterial pathologies such as thrombotic diseases (myocardial infarction, angina pectoris, cerebral vascular accidents, arteritis of the lower limbs etc.) and hypertension.

### Inhibition of the Expression of Aortic Vascular Cell Adhesion Molecule 1 (VCAM-1) and of Renal Fibronectin In Vivo

Four groups of 9 mice deficient in apolipoprotein E (ApoE^{-/-}, spontaneously developing atheroma plaques in their aortas) were used in this study. At the age of 8 weeks, the mice are made diabetic by 5 intraperitoneal injections of 70 mg/kg of streptozotocin over 5 days. At the ninth week, the animals are divided into four groups: an untreated control group, a group treated with terutroban (compound A) sodium salt (1 mg/kg/day in the food), a group treated with perindopril (compound B) tert-butylamine salt (0.1 mg/kg/day in the drinking water), and a group treated with the association of terutroban (compound A) sodium salt (1 mg/kg/day in the food) and perindopril (compound B) tert-butylamine salt (0.1 mg/kg/day in the drinking water).

For the expression of renal fibronectin, the mice are treated for 6 weeks and then sacrificed after anaesthesia using isoflurane.

For the expression of aortic VCAM-1, the mice are treated for 13 weeks and then sacrificed. The aortas and the right-side kidneys are removed, dissected and frozen in liquid nitrogen. The tissues are cryo-ground and total RNA is extracted using the RNeasy® micro kit (Qiagen). Reverse transcription is then performed on 1 μg of total RNA using the Superscript™ III first-strand cDNA synthesis kit (Invitrogen). Expression of aortic VCAM-1 and expression of renal fibronectin are quantified by real-time PCR and normalised with respect to 3 reference genes: β-actin, hypoxanthine-guanine phosphoribosyl transferase (HPRT) and glyceraldehyde phosphate dehydrogenase (GAPDH). The IQ™ SYBR® Green supermix kit (Biorad) is used, with 2 μl of cDNA and 150 nM of each primer. The samples are denatured for 5 minutes at 95° C. and amplified for 40 cycles in accordance with the following protocol: denaturation for 20 seconds at 95° C. and hybridisation and elongation for 1 minute at 52° C. for fibronectin, at 54° C. for VCAM-1, β-actin and HPRT, and at 56° C. for GAPDH. The threshold cycle (defined as the cycle for which the fluorescence is considered to be significantly higher than the background noise) for aortic VCAM-1 and renal fibronectin of the untreated animals is normalised with respect to the reference genes (and considered to be 100%) and then compared to that of the treated animals.

The specific primers used are as follows:

VCAM-1:

(SEQ ID NO. 3)

5′-AGA GCA GAC TTT CTA TTT CAC-3′(sense)
and

(SEQ ID NO. 4)

5′-CCA TCT TCA CAG GCA TTT C-3′(antisense);
Fibronectin:

(SEQ ID NO. 5)

5′-TGA CAA ATA CAC TGG GAA C-3′(sense)
and

(SEQ ID NO. 6)

5′-GCC AAT CTT GTA GGA CTG-3′(antisense);
β-actin:

(SEQ ID NO. 7)

5′-AAG ACC TCT ATG CCA ACA CAG-3′(sense)
and

(SEQ ID NO. 8)

5′-AGC CAC CGA TCC ACA CAG-3′(antisense);
HPRT:

(SEQ ID NO. 9)

5′-AGC TAC TGT AAT GAT CAG TCA ACG-3′(sense)
and

(SEQ ID NO. 10)

5′-AGA GGT CCT TTT CAC CAG CA-3′(antisense);
GAPDH:

(SEQ ID NO. 11)

5′-GCC TTC CGT GTT CCT ACC C-3′(sense)
and

(SEQ ID NO. 12)

5′-TGC CTG CTT CAC CAC CTT-3′(antisense).

The activity of the compounds terutroban (compound A) sodium salt, perindopril (compound B) tert-butylamine salt, and of their association is assessed by comparing the levels of expression of aortic VCAM-1 and renal fibronectin to those of the untreated animals (considered to be 100%).

### Renal Fibronectin:

Treating the mice with terutroban (compound A) sodium salt or perindopril (compound B) tert-butylamine salt on their own has no significant effect on expression of the renal fibronectin gene (69±16% for terutroban (compound A) sodium salt and 86±9.9% for perindopril (compound B) tert-butylamine salt versus 100% without treatment, NS, single-factor ANOVA, with post-test Dunnett). When the mice are treated with the association of terutroban (compound A) sodium salt and perindopril (compound B) tert-butylamine salt, inhibition of expression of the fibronectin gene is then observed (43±9.1% versus 100% without treatment, P<0.01, single-factor ANOVA, with post-test Dunnett).

### Aortic VCAM-1:

Treatment of the mice with terutroban (compound A) sodium salt or perindopril (compound B) tert-butylamine salt on their own has no significant effect on expression of the aortic VCAM-1 gene (the residual expression is 88±22% for terutroban (compound A) sodium salt and 76±21% for perindopril (compound B) tert-butylamine salt versus 100% without treatment, NS, single-factor ANOVA, with post-test Dunnett). When the mice are treated with the association of terutroban (compound A) sodium salt and perindopril (compound B) tert-butylamine salt, inhibition of expression of the VCAM-1 gene is then observed (the residual expression is 27±6.2% versus 100% without treatment, P<0.01, single-factor ANOVA, with post-test Dunnett).

Treatment of the atheromatous and diabetic animals with the association terutroban (compound A) sodium salt/perindopril (compound B) tert-butylamine salt makes it possible therefore to clearly and significantly reduce the expression of VCAM-1 in the aorta and of fibronectin in the kidney, this being the case relative to untreated animals or animals treated with terutroban (compound A) sodium salt or perindopril (compound B) tert-butylamine salt on their own. The results show very clearly that administration of these two compounds in association makes it possible to obtain a substantial synergistic effect which was entirely unexpected.

This test demonstrates the inhibitory activity on the expression of adhesion molecules and the inhibitory activity on renal fibronectin of the association terutroban (compound A)/perindopril (compound B) and, therefore, potential for the treatment of arterial pathologies such as vascular complications associated with diabetes, hypertension, atherosclerosis, inflammation, metabolic syndrome associated with obesity, vascular complications associated with obesity, angina pectoris, arteritis of the lower limbs and cerebral vascular accidents. In view of the part played by adhesion molecules in venous disease, the association may also treat that disease.

1. A composition comprising a combination of compound (A) of formula (I), optionally in the form of an optical isomer or a pharmaceutically acceptable salt thereof, and an angiotensin-converting enzyme inhibitor or a pharmaceutically acceptable salt thereof:

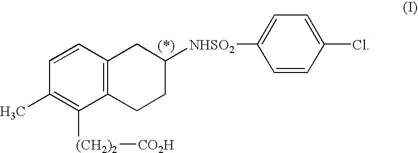

2. The composition of claim 1, wherein compound (A) is terutroban.

3. The composition of claim 1, wherein compound (A) is in the form of a sodium salt.

4. The composition of claim 1, wherein the angiotensin-converting enzyme inhibitor is perindopril, optionally in the form of its active metabolite perindoprilate; ramipril, optionally in the form of its active metabolite ramiprilate; enalapril, optionally in the form of its active metabolite enalaprilate; captopril, lisinopril, delapril, fosinopril, quinapril, spirapril, imidapril, trandolapril; optionally in the form of its active metabolite trandolaprilate, benazepril, cilazapril, temocapril, alacepril, ceronapril, moveltipril or moexipril, or an addition salt thereof with a pharmaceutically acceptable acid or base.

5. The composition of claim 1, wherein the angiotensin-converting enzyme inhibitor is perindopril or an addition salt thereof with a pharmaceutically acceptable acid or base.

6. The composition of claim 5, wherein the perindopril is in the form of a tert-butylamine or arginine salt.

7. A pharmaceutical composition comprising as active ingredient the composition of claim 1, in combination with one or more inert, pharmaceutically acceptable excipients or carriers.

8. The pharmaceutical composition of claim 7, wherein the angiotensin converting enzyme is perindopril, and wherein the composition comprises from 60 to 90% by weight of compound (A) and from 10 to 40% by weight of perindopril.

9. The pharmaceutical composition of claim 7, which is administered to a human or animal subject for the treatment of vascular complications associated with diabetes, with atherothrombotic diseases, with hyperlipidaemia, with hypertension, with chronic venous diseases, with inflammation, with metabolic syndrome associated with obesity, or with cancer.

10. The pharmaceutical composition of claim 9, which is administered to a human or animal subject for the treatment of cardiovascular and cerebrovascular complications associated with diabetes, with atherothrombotic diseases, with hyperlipidaemia, with hypertension, with chronic venous diseases, with inflammation, with metabolic syndrome associated with obesity, or with cancer.

11. The pharmaceutical composition of claim 9, for administration to a human or animal subject for the treatment of myocardial infarction, cerebral vascular accidents, aortic aneurysms or arteritis of the lower limbs.

12. The pharmaceutical composition of claim 9, for administration to a human or animal subject for the treatment of nephropathy associated with diabetes, with hypertension or with inflammatory diseases.

13. The pharmaceutical composition of claim 9, for administration to a human or animal subject for the treatment of diabetic retinopathy or nephropathy.

14. The pharmaceutical composition of claim 7, for administration to a human or animal subject for the treatment of dementias.

15. The pharmaceutical composition of claim 14, for administration to a human or animal subject for the treatment of Alzheimer's disease or vascular dementias.

16. A method for the treatment of vascular complications associated with diabetes, atherothrombotic diseases, hyperlipidaemia, hypertension, chronic venous diseases, inflammation, metabolic syndrome associated with obesity, or cancer, comprising the step of administering to a human or animal subject, the composition of claim 1.

17. The method of claim 16, wherein the vascular complications are selected from cardiovascular complications and cerebrovascular complications, wherein the cardiovascular and cerebrovascular complications are associated with diabetes, atherothrombotic diseases, hyperlipidaemia, hypertension, chronic venous diseases, inflammation, metabolic syndrome associated with obesity, or cancer.

18. The method of claim 16, wherein the atherothrombotic diseases are selected from myocardial infarction, cerebral vascular accidents, aortic aneurysms, and arteritis of the lower limbs.

19. The method of claim 16, wherein the vascular complications are nephropathy associated with diabetes, with hypertension or with inflammatory diseases.

20. The method of claim 16, wherein the vascular complications are selected from diabetic retinopathy and nephropathy.

21. The method of claim 16, wherein the vascular complications are associated with diabetes.

22. The method of claim 16, wherein the vascular complications are associated dementias.

23. The method of claim 22, wherein the dementias are selected from Alzheimer's disease and vascular dementias.
